(19) United States
(12) Patent Application Publication
(10) Pub. No.: US 2017/0299609 A1
(43) Pub. Date: Oct. 19, 2017

(54) TREATMENT OF AMYOTROPHIC LATERAL SCLEROSIS WITH SK CHANNEL ACTIVATORS

(71) Applicant: Wright State University, Dayton, OH (US)

(72) Inventor: Sherif Elbasiouny, Dayton, OH (US)

(73) Assignee: Wright State University, Dayton, OH (US)

(21) Appl. No.: 15/490,094

(22) Filed: Apr. 18, 2017

Related U.S. Application Data
(60) Provisional application No. 62/323,861, filed on Apr. 18, 2016.

(51) Int. Cl. G01N 33/68; A61K 31/522; A61K 31/712; C12N 15/85

(52) U.S. Cl. CPC G01N 33/6872; A61K 31/712; C12N 15/8509; A61K 31/522; C12N 2015/8536; G01N 2500/04; C12N 2015/8527

(57) ABSTRACT

Methods for treating amyotrophic lateral sclerosis (ALS). The methods include administering to a subject in need thereof a therapeutically effective amount of at least one small conductance calcium-activated potassium (SK) channel activator or a pharmaceutically acceptable salt or solvate thereof. Pharmaceutical compositions for the treatment of ALS, including a therapeutically effective amount of at least one SK channel activator, or a pharmaceutically acceptable salt or solvate thereof, and at least one excipient, adjuvant, or pharmaceutically acceptable carrier.

### CROSS-REFERENCE TO RELATED APPLICATIONS

This application claims the benefit under 35 U.S.C. §119 of U.S. Provisional Patent Application No. 62/323,861, filed on Apr. 18, 2016, entitled, “Treatment of Amyotrophic Lateral Sclerosis with SK Channel Activators” (Docket WRU 0383 MA/40878.521), the contents of which are hereby incorporated by reference in their entirety.

### TECHNICAL FIELD

The present disclosure relates generally to treatment of amyotrophic lateral sclerosis and to pharmaceutical compositions for the treatment of amyotrophic lateral sclerosis.

### BACKGROUND

Amyotrophic lateral sclerosis (hereinafter, “ALS”), or Lou Gehrig disease, is a fatal, progressive neurodegenerative disease that affects nerve cells in the brain and spinal cord. Specifically, in ALS, motor neurons which innervate muscle fibers and control movement degenerate and die. When motor neurons die, the ability of the brain to initiate and control muscle movement is lost. The life expectancy of persons diagnosed with ALS is typically from about 3 to 5 years. Death in persons with ALS is typically the result of respiratory failure caused by the degeneration of respiratory motor neurons. Despite ongoing research into ALS, there has been little improvement in life expectancy.

Currently, there is only one drug approved by the Food and Drug Administration (hereinafter, “FDA”) to treat ALS, riluzole. Riluzole is effective to extend survival and/or time to tracheostomy in some persons with ALS. On average, riluzole extends survival of humans with ALS by about 3 months. However, there are many side effects associated with riluzole, such as, e.g., excessive weakness.

### SUMMARY

Provided herein is an entirely new paradigm for ALS treatment. In embodiments, methods for treating ALS are disclosed. The methods include administering to a subject in need thereof a therapeutically effective amount of at least one small conductance calcium-activated potassium (hereinafter, “SK”) channel activator.

In embodiments, pharmaceutical compositions for the treatment of ALS are disclosed. The pharmaceutical compositions include a therapeutically effective amount of at least one SK channel activator, or a pharmaceutically acceptable salt or solvate thereof, and at least one excipient, adjuvant, or pharmaceutically acceptable carrier.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 is a bar graph of transgenic, G93A, male ALS mice injected with Vehicle Solution, i.e., “Vehicle”, (n=13) or with N-Cyclohexyl-N-[2-(3,5-dimethyl-pyrazol-1-yl)-6-methyl-4-pyrimidinamine (i.e., CyPPA), i.e., “16 days”, (n=12) (daily intraperitoneal injection of 0.014 μg/kg) for 16 days starting at 5 days of age (i.e., P5) with respect to mean survival, i.e., “Age at death (days)”. Bars represent standard error of the mean (hereinafter, “SEM”) and * indicates significance at p<0.001;

FIG. 2 is a bar graph of transgenic, G93A, male ALS mice injected with Vehicle Solution, i.e., “Vehicle”, (n=13) or with CyPPA, i.e., “CyPPA” (n=12) (daily intraperitoneal injection of 0.014 μg/kg) for 16 days starting at P5 with respect to mean age of completion of rotarod testing, i.e., “Age of rotarod test completion (days)”. Bars represent SEM and * indicates significance at p<0.05;

FIG. 3 is graph of Age, i.e., “Days of Age”, of transgenic, G93A, male ALS mice injected with Vehicle Solution (n=13) or CyPPA (n=12) (daily intraperitoneal injection of 0.014 μg/kg) for 16 days starting at P5 with respect to mean motor performance over time, i.e., “Percent Complete”; and

FIG. 4 is a graph of Age, i.e., “Age (Days)”, of transgenic, G93A, male ALS mice injected with Vehicle Solution (n=14) or CyPPA (n=14) (daily intraperitoneal injection of 0.014 μg/kg) for 7 days starting at P90 with respect to percentage completion ratio in rotarod testing, i.e., “Rotarod Completion Ratio”. The black bars (between P92-P105, Bar A, and P112-P119, Bar B) represent the 22 day periods in which the average motor performance of the transgenic, G93A, male ALS mice injected with CyPPA was significantly better than the transgenic, G93A, male ALS mice treated with Vehicle.

### DETAILED DESCRIPTION

While the following terms are believed to be well understood by one of ordinary skill in the art, definitions are set forth to facilitate explanation of the presently-disclosed subject matter.

The terms “treat,” “treatment,” and “treating,” as used herein, refer to delaying acquisition, inhibiting development or progression of, stabilizing, causing regression of, and/or reducing the risk of developing and/or acquiring a disease, disorder, and/or symptom thereof.

Depending upon the context of use, the term “subject in need thereof” as used herein, refers to a subject at risk for developing ALS, a subject exhibiting symptoms associated with ALS, and/or a subject having ALS. Examples of a subject at risk for developing ALS include, but should not be limited to, a subject having various gene mutations which can lead to familial or inherited ALS, a subject having a chemical imbalance (such as, e.g., higher than a threshold level of glutamate around the nerve cells in the spinal fluid), a subject having a disorganized immune response (such as, e.g., a subject having an immune system which attacks some of the subject's normal cells, which can lead to death of nerve cells), and/or a subject having protein mishandling (such as, e.g., a subject having an accumulation of abnormal forms of mishandled proteins in nerve cells, which can destroy nerve cells). Examples of symptoms associated with ALS include, but should not be limited to, slurred speech, difficulty chewing, difficulty swallowing, difficulty speaking, difficulty breathing, and/or loss of motor function, such as, e.g., difficulty walking, limb weakness, difficulty keeping good posture, and/or twitching. With regard to a subject having ALS, diagnosis of ALS may be performed using standard diagnostic techniques for ALS, such as are known to those of ordinary skill in the art. Examples of standard diagnostic techniques for ALS include, but should not be limited to, electromyograms (i.e., EMGs), nerve conduction studies, magnetic resonance imaging (i.e., MRI), blood and urine tests, spinal taps (i.e., lumbar punctures), and/or muscle biopsies, such as are known to those of ordinary skill in the art.

The term “therapeutically effective amount” as used herein, refers to an amount necessary or sufficient to realize a desired biologic effect. The therapeutically effective amount may vary depending on a variety of factors known to those of ordinary skill in the art, including but not limited to, the particular composition being administered, the activity of the composition being administered, the size of the subject, the sex of the subject, the age of the subject, the general health of the subject, the timing and route of administration, the rate of excretion, the administration of additional medications, and/or the severity of the disease or disorder being treated. In some embodiments, the term therapeutically effective amount refers to the amount of the at least one SK channel activator necessary or sufficient to treat ALS. More specifically, in embodiments, the term therapeutically effective amount refers to the amount of the at least one SK channel activator necessary or sufficient to extend survival and/or to improve motor function of the subject relative to a control.

The terms “small conductance calcium-activated potassium channel activator” and “SK channel activator” as used herein, refer to a compound capable of increasing the current mediated through a small conductance calcium-activated potassium channel and/or increasing the number or expression of small conductance calcium-activated potassium channels on a cell membrane. In embodiments, an SK channel activator is a compound capable of increasing an ion flux out of a cell having a small conductance calcium-activated potassium channel relative to a control. In embodiments, an SK channel activator is a compound capable of increasing an ion flux out of a cell having an SK1 channel, an SK2 channel, an SK3 channel, and/or an SK4 channel relative to a control, such as, e.g., an untreated control. In other embodiments, an SK channel activator is a compound capable of increasing expression of a small conductance calcium-activated potassium channel of a cell relative to a control, such as, e.g., an untreated control.

The term “pharmaceutically acceptable” as used herein, refers to a pharmaceutically active agent and/or other agents/ingredients for use in a pharmaceutical composition which are not deleterious to a subject receiving the pharmaceutical composition and/or which are suitable for use in contact with the tissues of humans and lower animals without undue toxicity, irritation, allergic response, and the like commensurate with a reasonable benefit/risk ratio.

The terms “pharmaceutically acceptable salt” and “pharmaceutically acceptable salts” as used herein, refer to salts prepared from pharmaceutically acceptable non-toxic bases or acids including inorganic or organic bases and inorganic or organic acids. Salts derived from inorganic bases include: aluminum, ammonium, calcium, copper, ferric, ferrous, lithium, magnesium, manganic salts, manganous, potassium, sodium chloride, zinc, and the like. Salts derived from pharmaceutically acceptable organic non-toxic bases include: salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When a compound is basic, salts may be prepared from pharmaceutically acceptable non-toxic acids, including inorganic and organic acids. Such acids include: acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Thus, representative pharmaceutically acceptable salts include but are not limited to acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexyl-resorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. It will be understood that, as used herein, the compounds referred to herein are meant to also include the pharmaceutically acceptable salts.

The terms “improve,” “improving,” and “improvement,” as used herein, refer to the enhanced ability of ALS treated subjects to move and/or to stay active as compared to control ALS subjects. In humans, an improvement in motor function could be determined via a decline in the rate of loss of motor function in ALS treated patients as compared to control ALS patients.

The term “baseline level” as used herein, refers to a level of SK channel expression in a subject prior to administration of the at least one SK channel activator for treating ALS. The baseline level may be quantifiably determined via molecular determinations.

The term “defined treatment period” refers to a period of time in which treatment is administered. In embodiments, the treatment period is a definite period of time, such as, e.g., for about 3 months upon diagnosis of ALS, or for about a year upon diagnosis of ALS, in which at least one SK channel activator is administered. In embodiments, the defined treatment period does not extend over the entire progression of ALS, such as, e.g., from diagnosis and/or early stages to late stages and/or death. In embodiments, the defined treatment period is reduced relative to the period of time in which existing ALS treatments, such as, e.g., riluzole, are administered. In embodiments, the defined treatment period begins prior to diagnosis of ALS, such as, e.g., treatment administration in a subject at risk for developing ALS.

The term “carrier” as used herein, refers to a solid or liquid filler, diluent or encapsulating substance. These materials are well known to those skilled in the pharmaceutical arts. Some examples of the substances that can serve as pharmaceutical carriers include sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; stearic acid; magnesium stearate; calcium sulfate; vegetable oils, such as peanut oil, cottonseed oil, sesame oil, olive oil, corn oil and oil of theobroma; polyols, such as propylene glycol, glycerine, sorbitol, mannitol, and polyethylene glycol; agar; alginic acid; pyrogen-free water; isotonic saline; and phosphate buffer solutions, as well as other non-toxic compatible substances used in pharmaceutical formulations. Wetting agents and lubricants, such as sodium lauryl sulfate, as well as coloring agents, flavoring agents, tableting agents, and preservatives, can also be present. Formulation of the components into pharmaceutical compositions is done using conventional techniques.

Embodiments of the present disclosure relate to methods for treating ALS and to pharmaceutical compositions for treating ALS. Embodiments of the methods for treating ALS will now be described in detail. Thereafter, embodiments of the pharmaceutical compositions for treating ALS will be described in detail.

### I. Methods for Treating ALS

Methods for treating ALS are disclosed. In embodiments, the methods include administering to a subject in need thereof a therapeutically effective amount of at least one SK channel activator. In embodiments, ALS includes familial or inherited ALS and/or sporadic ALS.

In embodiments, administering the at least one SK channel activator is effective to treat ALS by at least one of extending survival of the subject or by improving motor function of the subject relative to a control. In some embodiments, the at least one SK channel activator is effective to treat ALS by at least one of extending survival of the subject or by improving motor function of the subject relative to a control. In embodiments, the at least one SK channel activator is effective to increase expression of at least one SK channel in the subject relative to a baseline level or to a control. In embodiments, the at least one SK channel is an SKI channel, an SK2 channel, an SK3 channel, or an SK4 channel. In some embodiments, the control is a control population of subjects having ALS and/or a control population of cells known to those of skill in the art for studying ALS.

In embodiments, the at least one SK channel activator is administered systemically. Systemic administration of the at least one SK channel activator may be chosen from sublingual, subcutaneous, intravenous, intramuscular, intranasal, intrathecal, intraperitoneal, percutaneous, intranasal, enteral, or a combination thereof. In one or more embodiments, the at least one SK channel activator is administered orally.

In embodiments, the methods for treating ALS include administering at least one SK channel activator, or pharmaceutically-acceptable salts or solvates thereof, to a subject in need thereof, wherein the subject is a mammal. In one or more particular embodiments, the subject is a mammal chosen from humans, non-human primates, canines, felines, murines, bovines, equines, porcines, and lagomorphs. In some embodiments, the subject is a mouse or a human.

In embodiments, the methods for treating ALS include administering the at least one SK channel activator in a dose of from about 0.01 μg/kg to about 30 mg/kg, or from about 0.05 μg/kg to about 20 mg/kg, or from about 0.1 μg/kg to about 10 mg/kg, or from about 1 μg/kg to about 1 mg/kg, or from about 10 μg/kg to about 0.5 mg/kg, or about 0.1 mg/kg. It is contemplated that such doses serve as non-limiting examples of suitable doses of the at least one SK channel activator for a subject in need thereof. In embodiments, the dose of the at least one SK channel activator is administered daily. In some embodiments, the at least one SK channel activator is administered at least once a day. In other embodiments, the at least one SK channel activator is administered at least two times a day, at least three times a day, at least four times a day, at least five times a day, and/or at least six times a day. In particular embodiments, the at least one SK channel activator is administered from about one to about three times a day.

In embodiments, the at least one SK channel activator is chosen from an SK1 channel activator, an SK2 channel activator, an SK3 channel activator, an SK4 channel activator, a pharmaceutically acceptable salt or solvate thereof, or a combination thereof. In some embodiments, the at least one SK channel activator is chosen from N-Cyclohexyl-N-[2-(3,5-dimethyl-pyrazol-1-yl)-6-methyl-4-pyrimidinamine (hereinafter, “CyPPA”), (4-Chloro-phenyl)-[2-(3,5-dimethyl-pyrazol-1-yl)-9-methyl-9H-purin-6-yl]-amine) (hereinafter, “NS13001”), 5,6-Dichloro-1-ethyl-1,3-dihydro-2H-benzimidazol-2-one (hereinafter, “DCEBIO”), 1-Ethyl-2-benzimidazolinone (hereinafter, “1-EBIO”), 4-[[[[(2-Methoxyphenyl)amino]carbonyl]oxy]methyl]-piperidinecarboxylic acid-1,1-dimethylethyl ester (hereinafter, “GW 542573X”), 6,7-Dichloro-1H-indole-2,3-dione 3-oxime (hereinafter, “NS 309”), 2-amino-6-trifluoromethylthio-benzothiazole (hereinafter, “SKA 19”), Naphtho[1,2-d]thiazol-2-ylamine (hereinafter, “SKA 31”), 5-methylnaphtho[1,2-d]thiazol-2-amine (hereinafter, “SKA 111”), 5-methylnaphtho[2,1-d]oxazol-2-amine (hereinafter, “SKA 121”), 5-chloro-3H-1,3-benzoxazol-2-one (hereinafter, “Chlorzoxazone”), a pharmaceutically acceptable salt or solvate thereof, or a combination thereof. These SK channel activators are depicted in Table 1 below. In some embodiments, the at least one SK channel activator is chosen from CyPPA, NS13001, SKA-19, a pharmaceutically acceptable salt or solvate thereof, or a combination thereof. In other embodiments, the at least one SK channel activator is CyPPA or a pharmaceutically acceptable salt or solvate thereof. In embodiments, the at least one SK channel activator is CyPPA or a pharmaceutically acceptable salt or solvate thereof, which is effective to extend survival of the subject and/or to improve motor function of the subject relative to a control.

TABLE 1
SK Channel Activators

| Reference | Chemical Structure | Chemical Name |
|---|---|---|
| CyPPA | 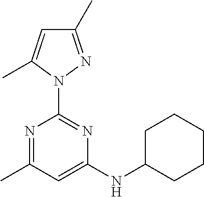 [SMILES: Cc1cc(NC2CCCCC2)nc(-n2nc(C)cc2C)n1] | N-Cyclohexyl-N-[2-(3,5- dimethyl-pyrazol-1-yl)-6- methyl-4-pyrimidinamine |
| NS13001 | 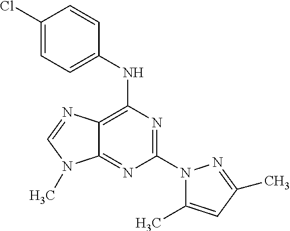 [SMILES: Cc1cc(C)n(-c2nc(Nc3ccc(Cl)cc3)c3ncn(C)c3n2)n1] | (4-Chloro-phenyl)-[2- (3,5-dimethyl-pyrazol-1- yl)-9-methyl-9H-purin-6- yl]-amine) |
| DCEBIO | 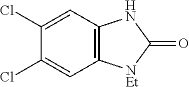 | 5,6-Dichloro-1-ethyl-1,3- dihydro-2H-benzimidazol- 2-one |
| 1-EBIO | 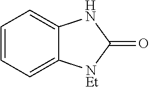 | 1-Ethyl-2- benzimidazolinone |
| GW 542573X | 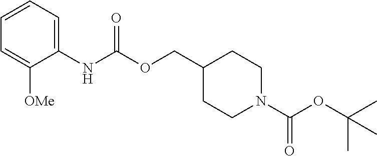 [SMILES: COc1ccccc1NC(=O)OCC1CCN(C(=O)OC(C)(C)C)CC1] | 4-[[[[(2- Methoxyphenyl)amino]car- bonyl]oxy]methyl]- piperidinecarboxylic acid- 1,1-dimethylethyl ester |
| NS 309 | 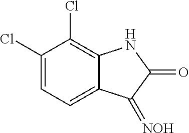 | 6,7-Dichloro-1H-indole- 2,3-dione 3-oxime |
| SKA-19 | 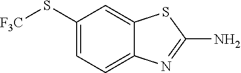 [SMILES: Nc1nc2ccc(SC(F)(F)F)cc2s1] | 2-amino-6- trifluoromethylthio- benzothiazole |
| SKA 31 | 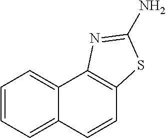 [SMILES: Nc1nc2c(ccc3ccccc32)s1] | Naphtho[1,2-d]thiazol-2- ylamine |
| SKA-111 | 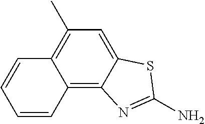 | 5-methylnaphtho[1,2- d]thiazol-2-amine |
| SKA-121 | 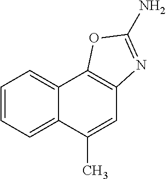 | 5-methylnaphtho[2,1- d]oxazol-2-amine |
| Chlor- zoxazone | 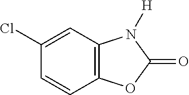 [SMILES: O=c1[nH]c2cc(Cl)ccc2o1] | 5-chloro-3H-1,3- benzoxazol-2-one |

In embodiments, the methods for treating ALS further include monitoring disease development and/or progression and repeating administration of the at least one SK channel activator or pharmaceutically-acceptable salts or solvates thereof one or more times, thereby treating ALS. Development and/or progression of ALS may be monitored in a variety of ways known to the skilled clinician. For example, development and/or progression of ALS may be monitored via characterizing the rate of loss of motor function. In embodiments, the methods for treating ALS further include monitoring disease development and/or progression and repeating administration of the at least one SK channel activator or pharmaceutically-acceptable salts or solvates thereof one or more times, thereby treating ALS. Successive rounds of administering the at least one SK channel activator coupled with monitoring development and/or progression of ALS may be necessary in order to achieve the desired treatment of ALS.

In embodiments, the at least one SK channel activator is administered in a pharmaceutical composition including at least one or an excipient, adjuvant, or pharmaceutically acceptable carrier. The pharmaceutical composition is as described in greater detail subsequently.

Embodiments of the methods for treating ALS have been described in detail. Embodiments of pharmaceutical compositions for treating ALS will now be described in detail.

### II. Pharmaceutical Compositions for Treating ALS

Pharmaceutical compositions for the treatment of ALS are disclosed. In embodiments, pharmaceutical compositions including a therapeutically effective amount of at least one SK channel activator or a pharmaceutically acceptable salt or solvate thereof, and at least one excipient, adjuvant, or pharmaceutically acceptable carrier, are disclosed. The at least one SK channel activator of the pharmaceutical composition is as previously described.

Examples of suitable excipients include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol®, and vegetable oils. Examples of suitable adjuvants include inorganic compounds (e.g., aluminum hydroxide, aluminum phosphate, calcium phosphate hydroxide, and beryllium), mineral oil (e.g., paraffin oil), bacterial products (e.g., killed bacteria Bordetelle pertussis, Mycobacterium bovis, and toxoids), nonbacterial organics (e.g., squalene and thimerosal), delivery systems (e.g., detergents (Quil A)), cytokines (e.g., IL-1, IL-2, and IL-12), and combinations (e.g., Freund's complete adjuvant, Freund's incomplete adjuvant). Examples of pharmaceutically acceptable carriers include a wide range of known diluents (i.e., solvents), fillers, extending agents, binders, suspending agents, disintegrates, surfactants, lubricants, wetting agents, preservatives, stabilizers, antioxidants, antimicrobials, buffering agents and the like commonly used in this field. Such carriers may be used singly or in combination according to the form of the pharmaceutical preparation. In further embodiments, a preparation resulting from the inclusion of a pharmaceutically acceptable carrier may incorporate, if necessary, one or more solubilizing agents, buffers, preservatives, colorants, perfumes, flavorings and the like, as widely used in the field of pharmaceutical preparation. Examples of suitable preservatives, stabilizers, antioxidants, antimicrobials, and buffering agents include BHA, BHT, citric acid, ascorbic acid, tetracycline, and the like. Cream or ointment bases useful in formulation include lanolin, Silvadene® (Marion), Aquaphor® (Duke Laboratories).

A pharmaceutical composition for the treatment of ALS may be prepared according to methods known in the pharmaceutical field using a pharmaceutically acceptable carrier. For example, oral forms such as tablets, capsules, granules, pills and the like are prepared according to known methods using excipients such as saccharose, lactose, glucose, starch, mannitol and the like; binders such as syrup, gum arabic, sorbitol, tragacanth, methylcellulose, polyvinylpyrrolidone and the like; disintegrates such as starch, carboxymethylcellulose or the calcium salt thereof, microcrystalline cellulose, polyethylene glycol and the like; lubricants such as talc, magnesium stearate, calcium stearate, silica and the like; and wetting agents such as sodium laurate, glycerol and the like.

Injections, solutions, emulsions, suspensions, syrups and the like may be prepared according to known methods suitably using solvents for dissolving the at least one SK channel activator, such as ethyl alcohol, isopropyl alcohol, propylene glycol, 1,3-butylene glycol, polyethylene glycol, sesame oil and the like; surfactants such as sorbitan fatty acid ester, polyoxyethylenesorbitan fatty acid ester, polyoxyethylene fatty acid ester, polyoxyethylene of hydrogenated castor oil, lecithin and the like; suspending agents such as cellulose derivatives including carboxymethylcellulose sodium, methylcellulose and the like, natural gums including tragacanth, gum arabic and the like; and preservatives such as parahydroxybenzoic acid esters, benzalkonium chloride, sorbic acid salts and the like.

In some embodiments, the pharmaceutical composition for the treatment of ALS includes a packaging material suitable for the pharmaceutical composition and instructions for use of the pharmaceutical composition for the treatment of ALS. In particular embodiments, the pharmaceutical composition for the treatment of ALS is provided for administration to a subject in unit dose and/or multi-dose containers, e.g., vials and/or ampoules. In specific embodiments, the pharmaceutical composition for the treatment of ALS is provided for administration to a subject in a device including a reservoir. In further specific embodiments, the pharmaceutical composition for the treatment of ALS is provided for administration to a subject in a device including a reservoir which is a vial, wherein the device is a syringe.

The pharmaceutical compositions for the treatment of ALS as described herein may be administered to a subject in need thereof in accordance with the methods for treating ALS, as described previously.

Embodiments of the pharmaceutical compositions for the treatment of ALS have been described in detail.

### Examples

The following non-limiting examples illustrate the methods of the present disclosure.

### Example 1: Characterization of the Effect of SK Channel Activators Prior to ALS Onset to Extend Survival and Improve Motor Function in Mice

Experimental Protocol and Results.

Based on data from computer simulations, electrophysiology, and immunohistochemistry, it was discovered that SK channels in motorneuron cells were downregulated in the G93A high expressor line of transgenic ALS mice with a B6SJL background. Thus, the effect of SK channel activators on survival and motor function of the G93A high expressor line of transgenic male ALS mice with a B6SJL background (hereinafter, “transgenic ALS mice”) was characterized. The transgenic ALS mice carry a mutation of the superoxide dismutase 1 gene; as a result, the transgenic ALS mice develop a neurodegenerative disease which closely mimics ALS in humans. In this experiment, CyPPA (n=13) or vehicle solution (i.e., saline+11% DMSO, n=12) was administered early to the transgenic ALS mice for sixteen (16) days (between ages P5 and P20) via daily intraperitoneal (i.e., IP) injection. CyPPA was administered at a dose of 0.014 μg/kg in both experiments.

In this experiment, the effect of CyPPA on survival of the transgenic ALS mice was assessed by determining when the transgenic ALS mice met end point criteria, i.e., full paralysis of both hind limbs and failing to right themselves 30 seconds after being turned on their backs. Additionally, the effect of CyPPA on motor function of the transgenic ALS mice was assessed by daily measuring from the age of P85 to the end stage using the rotarod machine. More specifically, the transgenic ALS mice were placed on a rotating rod for four minutes at a speed of 5 rotations per minute (i.e., RPM). The speed was increased to 25 RPM and the transgenic ALS mice were deemed unable to complete the task if they could complete only five percent (i.e., 12 seconds) of the task (i.e., the end stage). Rotarod performance was assessed to detect onset of symptoms associated with the neurodegenerative disease mimicking ALS in humans in the transgenic ALS mice and/or to monitor disease progression in the transgenic ALS mice, such as, e.g., via characterizing the rate of loss of motor function.

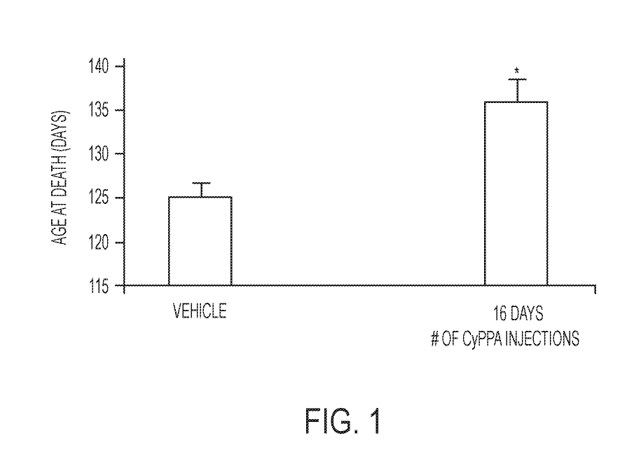

As shown in FIG. 1, the transgenic ALS mice injected with CyPPA for 16 days had longer survival by 10 days relative to control transgenic ALS mice injected with a vehicle solution. More specifically, the mean survival of the transgenic ALS mice injected with CyPPA for 16 days was 135.72 days and the mean survival of the control transgenic ALS mice injected with vehicle solution was 125.76 days. Thus, the transgenic ALS mice injected with CyPPA for 16 days exhibited an ˜8% increase in mean survival over the control transgenic mice injected with vehicle solution, p=0.0063. These data were highly statistically significant.

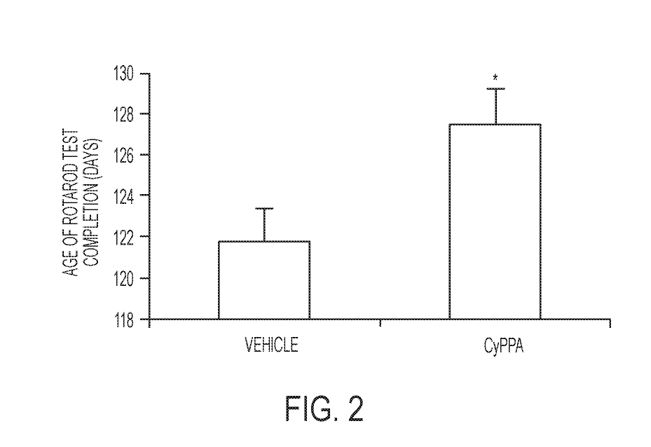

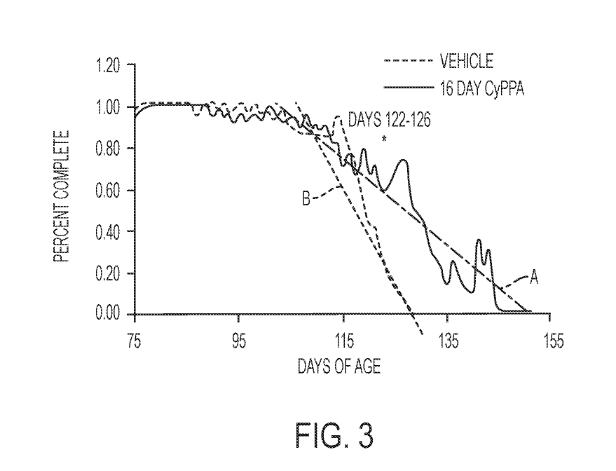

In addition to increased survival, the transgenic ALS mice injected with CyPPA for 16 days showed improved motor function. As shown in FIG. 2, the transgenic ALS mice injected with CyPPA for 16 days were able to complete the rotarod task 5 days longer than the transgenic ALS mice injected with vehicle solution. Further, as shown in FIG. 3, the transgenic ALS mice injected with CyPPA for 16 days exhibited an improved performance level as they aged, wherein they were able to stay on the rotarod significantly longer relative to the control transgenic ALS mice injected with vehicle solution. Specifically, the transgenic ALS mice injected with CyPPA for 16 days were able to stay on the rotarod for a longer time starting at 122 days of age and extending to 126 days of age, whereas the control transgenic ALS mice injected with vehicle solution stayed on the rotarod for a shorter time. Referencing FIG. 3, at day 126, no control transgenic ALS mice were able to stay on the rotarod for 12 seconds of the task.

Additionally, referencing FIG. 3, the rate of decline in motor function of the transgenic ALS mice injected with CyPPA for 16 days was reduced by ˜40% relative to the control transgenic ALS mice injected with vehicle solution (i.e., the slope of the transgenic ALS mice injected with CyPPA=0.0211 (i.e., line A) and the slope of the control transgenic ALS mice injected with vehicle solution=0.0349 (i.e., line B), p=0.0017 in FIG. 3). Referencing the dotted line in FIG. 3, such reduction indicates that the rate of ALS disease progression was reduced (p<0.05).

### Example 2: Characterization of the Effect of SK Channel Activators at ALS Onset to Extend Survival and Improve Motor Function in Mice

Experimental Protocol and Results.

Because symptoms of ALS in the transgenic ALS mice emerge around P90, the effect of administering CyPPA at P90 was characterized. Without being bound by the theory, it is believed that administration of CyPPA in the transgenic ALS mice at ALS onset would correlate to beginning treatment in humans upon diagnosis of ALS. In this experiment, CyPPA (n=14) or vehicle solution (i.e., saline+11% DMSO; n=14) was administered to the transgenic ALS mice for seven (7) days (between ages P90 and P96) via daily IP injection. CyPPA was administered at a dose of 0.014 μg/kg.

In this experiment, the effect of CyPPA on survival of the transgenic ALS mice was assessed by determining when the transgenic ALS mice met end point criteria, i.e., full paralysis of both hind limbs and failing to right themselves 30 seconds after being turned on their backs. Additionally, the effect of CyPPA on motor function of the transgenic ALS mice was assessed by daily measuring from the age of P85 to the end stage using the rotarod machine. More specifically, the transgenic ALS mice were placed on a rotating rod for four minutes at a speed of 5 rotations per minute (i.e., RPM). The speed was increased to 25 RPM and the transgenic ALS mice were deemed unable to complete the task if they could complete only five percent (i.e., 12 seconds) of the task (i.e., the end stage).

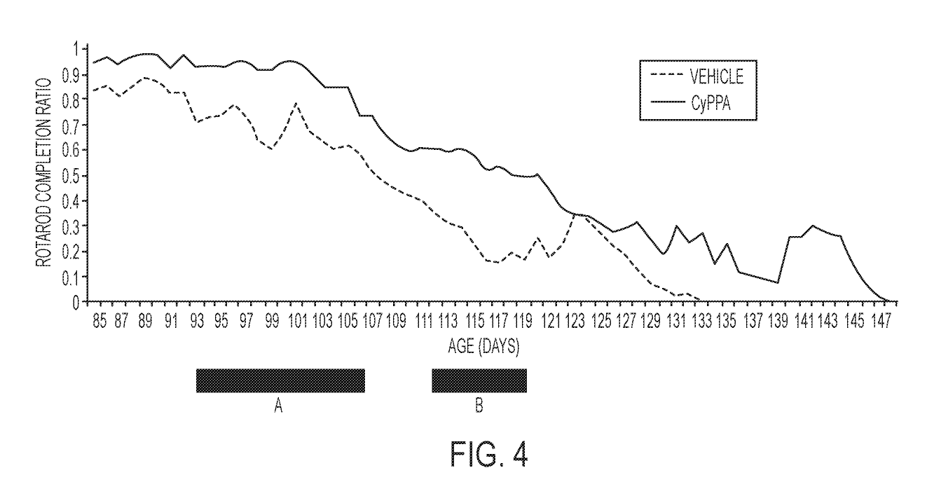

Referencing FIG. 4, there were significant differences in rotarod performance in the transgenic ALS mice injected with CyPPA for 7 days relative to control transgenic male injected with vehicle solution. Specifically, referencing FIG. 4, at most days tested, a larger percentage of the transgenic ALS mice injected with CyPPA were able to complete the rotarod task (i.e., by staying on the rotarod for more than 12 seconds) than the control transgenic ALS mice injected with vehicle solution. Referencing the days above the black bars of FIG. 4 (i.e., from 92 days of age extending to 105 days of age (i.e., Bar A) and from 112 days of age extending to 119 days of age (i.e., Bar B)), the average motor function of the transgenic ALS mice injected with CyPPA was significantly better than the control transgenic ALS mice injected with vehicle solution.

It is believed that FIGS. 1-4 demonstrate that the SK channel activator CyPPA has a beneficial effect on the survival and motor function of the transgenic ALS mice when treated at early or late time points in ALS disease. Without being bound by the theory, it is believed that such effect indicates that SK channel activators influence ALS disease progression. Further, it is believed that such effects in G93A high expressor line of transgenic ALS mice with a B6SJL background would correlate with treatment in humans, wherein the G93A high expressor line of transgenic ALS mice with a B6SJL background was employed in initial experimentation with riluzole, which translated to successful human treatment of ALS.

All documents cited are incorporated herein by reference; the citation of any document is not to be construed as an admission that it is prior art with respect to the present disclosure.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present disclosure have been illustrated and described, it would be obvious to one skilled in the art that various other changes and modifications can be made without departing from the spirit and scope of the disclosure. It is therefore intended to cover in the appended claims all such changes and modifications that are within the scope of this disclosure.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all technical and scientific terms used herein have the same meaning as commonly understood by one of ordinary skill in the art to which the claimed subject matter belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise. For example, reference to “a cell-permeable peptides” may include both reference to a single cell-permeable peptide and reference to a plurality of cell-permeable peptides.

What is claimed is:

1. A method for treating amyotrophic lateral sclerosis (ALS), the method comprising administering to a subject in need thereof a therapeutically effective amount of at least one small conductance calcium-activated potassium (SK) channel activator or a pharmaceutically acceptable salt or solvate thereof.

2. The method of claim 1, wherein the at least one SK channel activator is an SK1 channel activator, an SK2 channel activator, an SK3 channel activator, an SK4 channel activator, a pharmaceutically acceptable salt or solvate thereof, or a combination thereof.

3. The method of claim 1, wherein the at least one SK channel activator is chosen from N-Cyclohexyl-N-[2-(3,5-dimethyl-pyrazol-1-yl)-6-methyl-4-pyrimidinamine (CyPPA), (4-Chloro-phenyl)-[2-(3,5-dimethyl-pyrazol-1-yl)-9-methyl-9H-purin-6-yl]-amine) (NS13001), 5,6-Dichloro-1-ethyl-1,3-dihydro-2H-benzimidazol-2-one (DCEBIO), 1-Ethyl-2-benzimidazolinone (1-EBIO), 4-[[[[(2-Methoxyphenyl)amino]carbonyl]oxy]methyl]-piperidinecarboxylic acid-1,1-dimethylethyl ester (GW 542573X), 6,7-Dichloro-1H-indole-2,3-dione 3-oxime (NS309), 2-amino-6-trifluoromethylthio-benzothiazole (SKA 19), Naphtho[1,2-d]thiazol-2-ylamine (SKA 31), 5-methylnaphtho[1,2-d]thiazol-2-amine (SKA 111), 5-methylnaphtho[2,1-d]oxazol-2-amine (SKA 121), 5-chloro-3H-1,3-benzoxazol-2-one (Chlorzoxazone), a pharmaceutically acceptable salt or solvate thereof, or a combination thereof.

4. The method of claim 1, wherein the at least one SK channel activator is chosen from N-Cyclohexyl-N-[2-(3,5-dimethyl-pyrazol-1-yl)-6-methyl-4-pyrimidinamine (CyPPA), (4-Chloro-phenyl)-[2-(3,5-dimethyl-pyrazol-1-yl)-9-methyl-9H-purin-6-yl]-amine) (NS13001), 2-Amino-6-trifluoromethylthio-benzothiazole (SKA-19), a pharmaceutically acceptable salt or solvate thereof, or a combination thereof.

5. The method of claim 1, wherein the at least one SK channel activator is N-Cyclohexyl-N-[2-(3,5-dimethyl-pyrazol-1-yl)-6-methyl-4-pyrimidinamine (CyPPA) or a pharmaceutically acceptable salt or solvate thereof.

6. The method of claim 1, wherein the at least one SK channel activator is effective to treat ALS by at least one of extending survival of the subject or improving motor function of the subject relative to a control.

7. The method of claim 1, wherein:
the at least one SK channel activator is N-Cyclohexyl-N-[2-(3,5-dimethyl-pyrazol-1-yl)-6-methyl-4-pyrimidinamine (CyPPA) or a pharmaceutically acceptable salt or solvate thereof, and
CyPPA or the pharmaceutically acceptable salt or solvate thereof is effective to improve motor function of the subject relative to a control.

8. The method of claim 1, wherein the at least one SK channel activator is effective to increase expression of at least one SK channel in the subject relative to a baseline level.

9. The method of claim 1, wherein the subject is a mammal.

10. The method of claim 1, wherein the subject is a mouse or a human.

11. The method of claim 1, wherein the at least one SK channel activator is administered systemically.

12. The method of claim 1, wherein the at least one SK channel activator is administered in a daily dose of about 0.01 μg/kg to about 30 mg/kg.

13. The method of claim 12, wherein the at least one SK channel activator is administered in a daily dose of about 0.01 μg/kg to about 0.05 μg/kg.

14. The method of claim 1, wherein the at least one SK channel activator is administered in a daily dose for a defined treatment period.

15. The method of claim 1, wherein the at least one SK channel activator is administered in a pharmaceutical composition comprising at least one of an adjuvant, an excipient, or a pharmaceutically acceptable carrier.

16. A pharmaceutical composition for the treatment of amyotrophic lateral sclerosis (ALS) comprising a therapeutically effective amount of at least one small conductance calcium-activated potassium (SK) channel activator, or a pharmaceutically acceptable salt or solvate thereof, and at least one excipient, adjuvant, or pharmaceutically acceptable carrier.

17. The pharmaceutical composition of claim 16, wherein the at least one SK channel activator is N-Cyclohexyl-N-[2-(3,5-dimethyl-pyrazol-1-yl)-6-methyl-4-pyrimidinamine (CyPPA).
